(19) United States
(12) Patent Application Publication
(10) Pub. No.: US 2024/0358223 A1
(43) Pub. Date: Oct. 31, 2024

(54) ENDOSCOPE SYSTEM, MEDICAL INFORMATION PROCESSING METHOD, AND MEDICAL INFORMATION PROCESSING PROGRAM

(71) Applicant: FUJIFILM Corporation, Tokyo (JP)

(72) Inventor: Yuya KIMURA, Kanagawa (JP)

(73) Assignee: FUJIFILM Corporation, Tokyo (JP)

(21) Appl. No.: 18/767,937

(22) Filed: Jul. 9, 2024

Related U.S. Application Data
(63) Continuation of application No. PCT/JP2022/045977, filed on Dec. 14, 2022.

(30) Foreign Application Priority Data
Jan. 19, 2022 (JP) 2022-006229

(51) Int. Cl. A61B 1/00

(52) U.S. Cl. CPC A61B 1/00039; A61B 1/00009

(57) ABSTRACT

An endoscope system according to an aspect of the present invention is an endoscope system including: a speech recognition device configured to receive input of speech and perform speech recognition; an endoscope configured to acquire a medical image of a subject; and a processor. The processor is configured to: cause the endoscope to capture time-series medical images of the subject; detect delimiters of results of the speech recognition during capturing of the time-series medical images; and group and record, in a recording apparatus, the results of the speech recognition, the results being obtained during a period from detection of one of the delimiters until detection of an other one of the delimiters corresponding to the one of the delimiters at a time later than a time at which the one of the delimiters is detected.

### CROSS-REFERENCE TO RELATED APPLICATIONS

The present application is a Continuation of PCT International Application No. PCT/JP2022/045977 filed on Dec. 14, 2022 claiming priority under 35 U.S.C § 119 (a) to Japanese Patent Application No. 2022-006229 filed on Jan. 19, 2022. Each of the above applications is hereby expressly incorporated by reference, in its entirety, into the present application.

### BACKGROUND OF THE INVENTION

### 1. Field of the Invention

The present invention relates to an endoscope system, a medical information processing method, and a medical information processing program for performing speech input and speech recognition on a medical image.

### 2. Description of the Related Art

In the technical field of examination or diagnosis support using a medical image, it is known to recognize speech that is input by a user and perform processing based on recognition results. In addition, it is known to display information that is input by speech (for example, see JP2013-106752A and JP2006-221583A).

### SUMMARY OF THE INVENTION

In a case where speech recognition is performed in an examination using a medical image, it is difficult to grasp a relationship between the recognition results by simply displaying or recording the recognition results. However, the related art such as JP2013-106752A or JP2006-221583A described above does not sufficiently take such a point into consideration.

The present invention has been made in view of such circumstances, and an object thereof is to provide an endoscope system, a medical information processing method, and a medical information processing program by which it is possible to easily record related results of speech recognition.

In order to achieve the above-described object, an endoscope system according to a first aspect of the present invention is an endoscope system including: a speech recognition device configured to receive input of speech and perform speech recognition; an endoscope configured to acquire a medical image of a subject; and a processor. The processor is configured to: cause the endoscope to capture time-series medical images of the subject; detect delimiters of results of the speech recognition during capturing of the time-series medical images; and group and record, in a recording apparatus, the results of the speech recognition, the results being obtained during a period from detection of one of the delimiters until detection of an other one of the delimiters corresponding to the one of the delimiters at a time later than a time at which the one of the delimiters is detected.

According to the first aspect, since the results of the speech recognition during the period until the detection of the other one of the delimiters corresponding to the one of the delimiters at the time later than the time at which the one of the delimiters is detected are grouped and recorded in the recording apparatus, it is possible to easily record related results of the speech recognition, and a user can easily grasp the related results of the speech recognition by referring to the record.

In an endoscope system according to a second aspect based on the first aspect, the processor is configured to cause a display device to display item information indicating an item to be subjected to speech recognition and a result of the speech recognition corresponding to the item information if the speech recognition is started.

In an endoscope system according to a third aspect based on the second aspect, the processor is configured to record, in the recording apparatus, the results of the speech recognition corresponding to one set of pieces of the item information as one group.

In an endoscope system according to a fourth aspect based on the second or third aspect, the processor is configured to: continue to display the item information and the result of the speech recognition from detection of the one of the delimiters until detection of the other one of the delimiters; and change a display manner of the item information and the result of the speech recognition on the display device if the other one of the delimiters is detected.

In an endoscope system according to a fifth aspect based on any one of the second to fourth aspects, the processor is configured to cause the display device to display the item information and the result of the speech recognition in real time.

In an endoscope system according to a sixth aspect based on any one of the second to fifth aspects, the item information includes at least one of diagnosis, findings, treatment, or hemostasis.

In an endoscope system according to a seventh aspect based on any one of the first to sixth aspects, the processor is configured to detect the one of the delimiters as a start delimiter of grouping and detect the other one of the delimiters as an end delimiter of the grouping.

In an endoscope system according to an eighth aspect based on the seventh aspect, the processor is configured to group the results of the speech recognition during a period from detection of the end delimiter until re-detection of the end delimiter at a time later than a time at which the end delimiter is detected.

In an endoscope system according to a ninth aspect based on the seventh or eighth aspect, the processor is configured to detect, as the end delimiter, at least one of an end of detection of a specific subject in the medical image, speech input of a first specific word/phrase to the speech recognition device, continuation of a non-input state of speech input to the speech recognition device for a determined time or more, completion of speech input to all items to be subjected to speech recognition, completion of speech input to a specific item among the items to be subjected to speech recognition, acquisition of information indicating that an insertion length and/or an insertion shape of the endoscope has changed by a determined value or more, or a start or stop of an operation by a user of the endoscope system via an operating device.

In an endoscope system according to a tenth aspect based on any one of the seventh to ninth aspects, the processor is configured to detect, as the start delimiter, at least one of a start of detection of a specific subject in the medical image, speech input of a second specific word/phrase to the speech recognition device, input by a user of the endoscope system via an operating device, a start of a discrimination mode for the specific subject, a start of output of a discrimination result for the specific subject, or a start of a measurement mode for the specific subject.

In an endoscope system according to an eleventh aspect based on the ninth or tenth aspect, the processor is configured to determine at least one of a lesion, a candidate lesion region, a landmark, or a post-treatment region as the specific subject.

In an endoscope system according to a twelfth aspect based on any one of the ninth to eleventh aspects, the processor is configured to recognize the specific subject by using an image recognizer generated by machine learning.

In an endoscope system according to a thirteenth aspect based on any one of the eighth to twelfth aspects, the processor is configured to cause an output device to output a message for encouraging speech input for the medical image if the start delimiter is detected.

In an endoscope system according to a fourteenth aspect based on any one of the first to thirteenth aspects, the processor is configured to cause an image selected from the medical images captured by the endoscope during a period from detection of the one of the delimiters until detection of the other one of the delimiters to be grouped and recorded together with the results of the speech recognition.

In an endoscope system according to a fifteenth aspect based on any one of the first to fourteenth aspects, the processor is configured to cause an image selected from frame images constituting the time-series medical images and/or an image selected from captured images captured separately from the time-series medical images to be grouped and recorded together with the results of the speech recognition.

In an endoscope system according to a sixteenth aspect based on any one of the first to fifteenth aspects, the processor is configured to cause a display device to display the time-series medical images and a different display device to display the results of the speech recognition.

In order to achieve the above-described object, a medical information processing method according to a seventeenth aspect is a medical information processing method to be executed by an endoscope system including: a speech recognition device configured to receive input of speech and perform speech recognition; an endoscope configured to acquire a medical image of a subject; and a processor. The processor is configured to: cause the endoscope to capture time-series medical images of the subject; detect delimiters of results of the speech recognition during capturing of the time-series medical images; and group and record, in a recording apparatus, the results of the speech recognition, the results being obtained during a period from detection of one of the delimiters until detection of an other one of the delimiters corresponding to the one of the delimiters at a time later than a time at which the one of the delimiters is detected. According to the seventeenth aspect, as in the first aspect, it is possible to easily record related results of the speech recognition. Note that the seventeenth aspect may have substantially the same configuration as the second to sixteenth aspects.

In order to achieve the above-described object, a medical information processing program according to an eighteenth aspect of the present invention is a medical information processing program causing an endoscope system to execute a medical information processing method, the endoscope system including: a speech recognition device configured to receive input of speech and perform speech recognition; an endoscope configured to acquire a medical image of a subject; and a processor. In the medical information processing method, the processor is configured to: cause the endoscope to capture time-series medical images of the subject; detect delimiters of results of the speech recognition during capturing of the time-series medical images; and group and record, in a recording apparatus, the results of the speech recognition, the results being obtained during a period from detection of one of the delimiters until detection of an other one of the delimiters corresponding to the one of the delimiters at a time later than a time at which the one of the delimiters is detected. According to the eighteenth aspect, as in the first and seventeenth aspects, it is possible to easily record related results of the speech recognition. Note that the eighteenth aspect may have substantially the same configuration as the second to sixteenth aspects. In addition, a non-transitory and tangible recording medium on which a computer-readable code of the medical information processing program according to these aspects is recorded can also be given as an aspect of the present invention.

According to the endoscope system, the medical information processing method, and the medical information processing program according to the present invention, it is possible to easily record related results of the speech recognition.

### BRIEF DESCRIPTION OF THE DRAWINGS

FIG. 1 is a diagram illustrating a schematic configuration of an endoscopic image diagnosis system according to a first embodiment;

FIG. 2 is a diagram illustrating a schematic configuration of an endoscope system;

FIG. 3 is a diagram illustrating a schematic configuration of an endoscope;

FIG. 4 is a diagram illustrating an example of a configuration of an end surface of a tip part;

FIG. 5 is a block diagram illustrating main functions of an endoscopic image generation apparatus;

FIG. 6 is a block diagram illustrating main functions of an endoscopic image processing apparatus;

FIG. 7 is a block diagram illustrating main functions of an image recognition processing unit;

FIG. 8 is a diagram illustrating a display example of a message for encouraging speech input;

FIG. 9 is a block diagram illustrating main functions of a tablet terminal;

FIG. 10 is a diagram illustrating another display example of a message for encouraging speech input;

FIG. 11 is a diagram illustrating a state in which results of speech recognition are grouped;

FIGS. 12A to 12C are diagrams illustrating examples in which a lesion information input box is displayed;

FIGS. 13A and 13B are diagrams illustrating examples in which a display manner of the lesion information input box is changed;

FIG. 14 is a diagram illustrating a state in which an image is grouped together with results of speech recognition;

FIG. 15 is another diagram illustrating a state in which images are grouped together with results of speech recognition;

FIG. 16 is a diagram illustrating a state in which results of speech recognition are grouped by using an end of detection of a lesion as an end delimiter;

FIG. 17 is a diagram illustrating a state in which results of speech recognition are grouped by using a change in shape and insertion length of the endoscope as an end delimiter;

FIG. 18 is a diagram illustrating a state in which results of speech recognition are grouped by using a specific word/phrase as a start delimiter;

FIG. 19 is a diagram illustrating a schematic configuration of an endoscope system according to a second embodiment;

FIG. 20 is a block diagram illustrating main functions of an endoscopic image generation apparatus according to the second embodiment;

FIG. 21 is a block diagram illustrating main functions of an endoscopic image processing apparatus according to the second embodiment; and

FIG. 22 is a diagram illustrating a state in which a lesion information input box is displayed on a display device in the second embodiment.

### DESCRIPTION OF THE PREFERRED EMBODIMENTS

Embodiments of an endoscope system, a medical information processing method, and a medical information processing program according to the present invention will be described. In the description, the accompanying drawings are referred to as necessary. Note that in the accompanying drawings, some components may be omitted for convenience of description.

### Endoscopic Image Diagnosis Support System

### First Embodiment

A case where the present invention is applied to an endoscopic image diagnosis support system will be described as an example. The endoscopic image diagnosis support system is a system that supports detection and discrimination of a lesion or the like in an endoscopy. In the following, an example will be described in which the present invention is applied to an endoscopic image diagnosis support system that supports detection and discrimination of a lesion or the like in lower digestive tract endoscopy (large intestine examination).

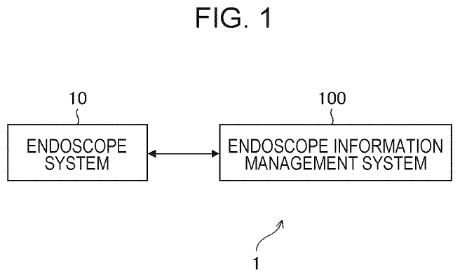

FIG. 1 is a block diagram illustrating a schematic configuration of the endoscopic image diagnosis support system.

As illustrated in FIG. 1, an endoscopic image diagnosis support system 1 (endoscope system) according to this embodiment has an endoscope system 10 (endoscope system) and an endoscope information management system 100. The endoscopic image diagnosis support system 1 may further have a user terminal.

### Endoscope System

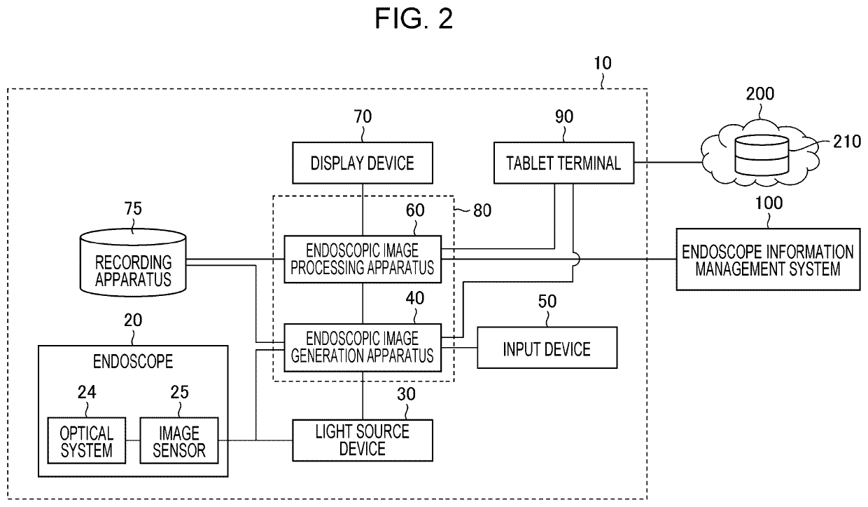

FIG. 2 is a block diagram illustrating a schematic configuration of the endoscope system 10.

The endoscope system 10 according to this embodiment is configured as a system capable of observation using special light (special-light observation) in addition to observation using white light (white-light observation). The special-light observation includes narrow-band light observation. The narrow-band light observation includes BLI observation (Blue laser imaging observation), NBI observation (Narrow band imaging observation; NBI is a registered trademark), LCI observation (Linked Color Imaging observation), and the like. Note that the special-light observation itself is a known technique, and thus, detailed description thereof is omitted.

As illustrated in FIG. 2, the endoscope system 10 according to this embodiment has an endoscope 20 (endoscope), a light source device 30, an endoscopic image generation apparatus 40 (processor), an endoscopic image processing apparatus 60 (processor), a display device 70 (display device), a recording apparatus 75 (recording apparatus), an input device 50, a tablet terminal 90 (processor, display device, recording apparatus), and the like. The endoscope 20 includes an optical system 24 and an image sensor 25 incorporated in a tip part 21A of an insertion part 21. Note that the endoscopic image generation apparatus 40 and the endoscopic image processing apparatus 60 constitute a medical information processing apparatus 80. In addition, the endoscope system 10 can access a database 210 on a cloud 200 via the tablet terminal 90.

### Endoscope

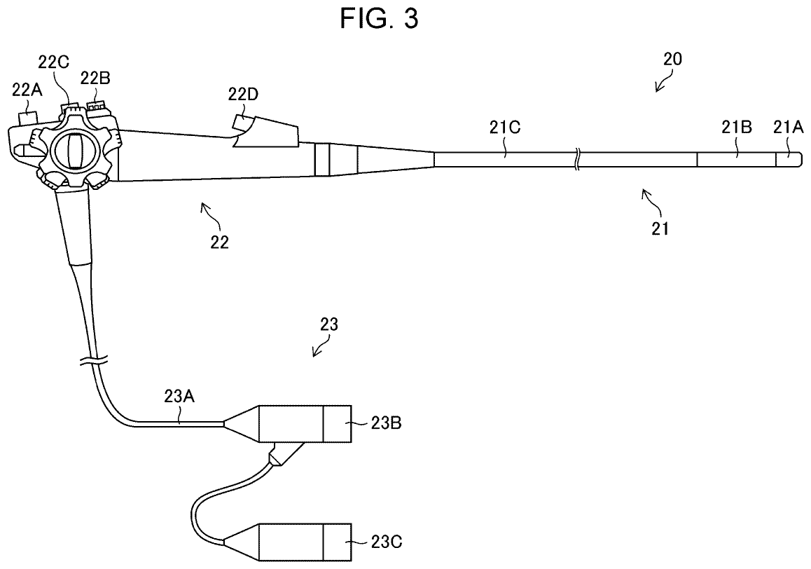

FIG. 3 is a diagram illustrating a schematic configuration of the endoscope 20.

The endoscope 20 according to this embodiment is an endoscope for a lower digestive tract. As illustrated in FIG. 3, the endoscope 20 is a flexible endoscope (electronic endoscope) and has the insertion part 21, an operating unit 22, and a connection part 23.

The insertion part 21 is a part to be inserted into a luminal organ (e.g., large intestine). The insertion part 21 is constituted by the tip part 21A, a bending part 21B, and a soft part 21C in order from the distal end side.

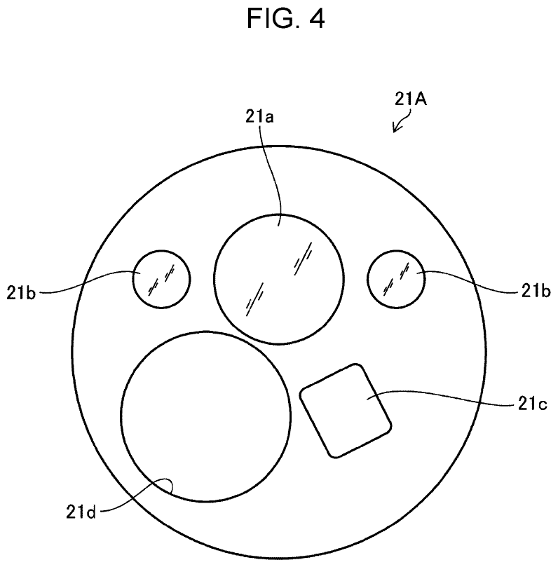

FIG. 4 is a diagram illustrating an example of a configuration of an end surface of the tip part.

As illustrated in FIG. 4, an observation window 21a, illumination windows 21b, an air/water supply nozzle 21c, a forceps outlet 21d, and the like are provided on the end surface of the tip part 21A. The observation window 21a is a window for observation. An image of the inside of a luminal organ of a subject is captured through the observation window 21a. An image is captured through the optical system 24 such as a lens and the image sensor 25 (image sensor; see FIG. 2) incorporated in the tip part 21A (part of the observation window 21a), and time-series images (moving images) and/or still images of the subject can be captured. As the image sensor, for example, a CMOS image sensor (Complementary Metal Oxide Semiconductor image sensor), a CCD image sensor (Charge Coupled Device image sensor), or the like is used. The illumination windows 21b are windows for illumination. The luminal organ is irradiated with illumination light through the illumination windows 21b. The air/water supply nozzle 21c is a nozzle for cleaning. A cleaning liquid and a drying gas are injected from the air/water supply nozzle 21c toward the observation window 21a. The forceps outlet 21d is an outlet for a treatment tool such as forceps. The forceps outlet 21d also functions as a suction port for sucking a body fluid or the like.

The bending part 21B is a part that bends in accordance with an operation of an angle knob 22A provided in the operating unit 22. The bending part 21B bends in four directions, which are up, down, left, and right.

The soft part 21C is an elongated part provided between the bending part 21B and the operating unit 22. The soft part 21C has flexibility.

The operating unit 22 is a part to be gripped by a surgeon to perform various operations. The operating unit 22 is provided with various operating members. As an example, the operating unit 22 is provided with the angle knob 22A for performing a bending operation of the bending part 21B, an air/water supply button 22B for performing an air supply/water supply operation, and a suction button 22C for performing a suction operation. In addition, the operating unit 22 is provided with an operating member (shutter button) for capturing a still image, an operating member for switching an observation mode, an operating member for switching ON and OFF of various support functions, and the like. In addition, the operating unit 22 is provided with a forceps insertion port 22D for inserting a treatment tool such as forceps. The treatment tool inserted from the forceps insertion port 22D is fed out from the forceps outlet 21d (see FIG. 4) at the distal end of the insertion part 21. As an example, the treatment tool includes biopsy forceps, a snare, and the like.

The connection part 23 is a part for connecting the endoscope 20 to the light source device 30, the endoscopic image generation apparatus 40, and the like. The connection part 23 is constituted by a cord 23A extending from the operating unit 22, a light guide connector 23B, a video connector 23C, and the like provided at the distal end of the cord 23A. The light guide connector 23B is a connector for connection to the light source device 30. The video connector 23C is a connector for connection to the endoscopic image generation apparatus 40.

### Light Source Device

The light source device 30 generates illumination light. As described above, the endoscope system 10 according to this embodiment is configured as a system capable of special-light observation in addition to normal white-light observation. Thus, the light source device 30 is configured to be capable of generating light (e.g., narrow-band light) corresponding to special-light observation in addition to normal white light. Note that, as described above, the special-light observation itself is a known technique, and thus, description of generation of the light and the like is omitted.

### Medical Information Processing Apparatus

### Endoscopic Image Generation Apparatus

The endoscopic image generation apparatus 40 (processor) integrally controls the operation of the entire endoscope system 10 together with the endoscopic image processing apparatus 60 (processor). The endoscopic image generation apparatus 40 includes, as its hardware configuration, a processor, a main storage (memory), an auxiliary storage (memory), a communication unit, and the like. That is, the endoscopic image generation apparatus 40 has a so-called computer configuration as its hardware configuration. The processor is constituted by, for example, a CPU (Central Processing Unit), a GPU (Graphics Processing Unit), an FPGA (Field Programmable Gate Array), a PLD (Programmable Logic Device), or the like. The main storage is constituted by, for example, a RAM (Random Access Memory) or the like. The auxiliary storage is constituted by, for example, a non-transitory and tangible recording medium such as a flash memory, a ROM (Read Only Memory), or an EEPROM (Electronically Erasable and Programmable Read Only Memory).

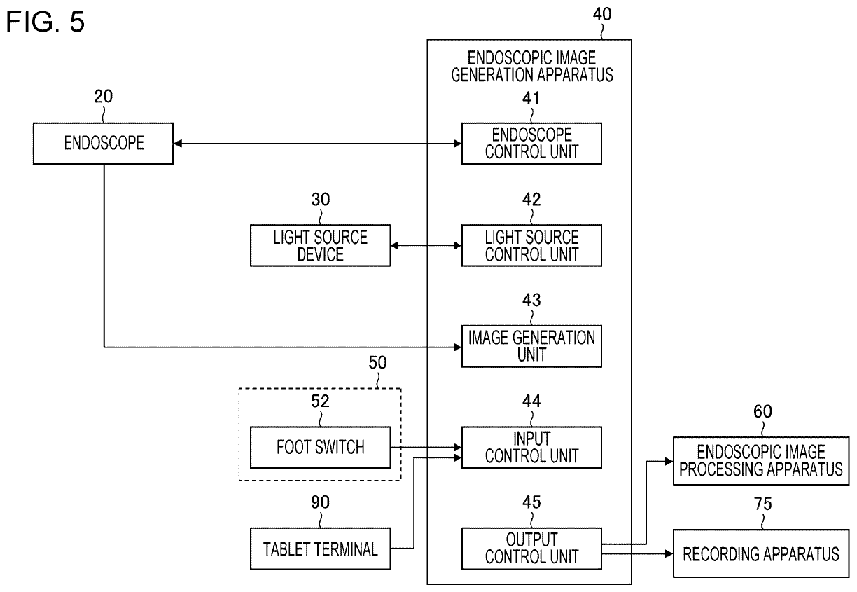

FIG. 5 is a block diagram illustrating main functions of the endoscopic image generation apparatus 40.

As illustrated in FIG. 5, the endoscopic image generation apparatus 40 has functions of an endoscope control unit 41, a light source control unit 42, an image generation unit 43, an input control unit 44, an output control unit 45, and the like. Various programs (which may include the medical information processing program according to the present invention or a part thereof) to be executed by the processor, various kinds of data necessary for control and the like, and the like are stored in the above-described auxiliary storage, and each function of the endoscopic image generation apparatus 40 is implemented by the processor executing these programs. The processor of the endoscopic image generation apparatus 40 is an example of a processor in the endoscope system and the medical information processing apparatus according to the present invention.

The endoscope control unit 41 controls the endoscope 20. The control of the endoscope 20 includes driving control of the image sensor 25, control of air supply and water supply, control of suction, and the like.

The light source control unit 42 controls the light source device 30. The control of the light source device 30 includes light emission control of a light source, and the like.

The image generation unit 43 generates a captured image (endoscopic image, medical image) based on a signal output from the image sensor 25 of the endoscope 20. The image generation unit 43 can generate a still image and/or a moving image (time-series medical images) as a captured image. The image generation unit 43 may perform various kinds of image processing on the generated image.

The input control unit 44 receives input of an operation and input of various kinds of information via the input device 50.

The output control unit 45 controls output of information to the endoscopic image processing apparatus 60. The information output to the endoscopic image processing apparatus 60 includes, in addition to an endoscopic image obtained by image capturing, various kinds of operation information input from the input device 50, and the like.

### Input Device

The input device 50 constitutes a user interface in the endoscope system 10 together with the display device 70. The input device 50 includes a foot switch 52 (operating device). The foot switch 52 is an operating device that is placed at the foot of a surgeon and is operated by a foot, and pressing of a pedal causes an operation signal (e.g., a signal for selecting a candidate for speech recognition or a signal indicating a start or end delimiter of grouping of results of speech recognition) to be output. Note that the foot switch 52 is controlled by the input control unit 44 of the endoscopic image generation apparatus 40 in this aspect, but is not limited to this aspect and may be controlled via the endoscopic image processing apparatus 60, the display device 70, or the like. In addition, the operating unit 22 of the endoscope 20 may be provided with an operating device (e.g., a button or a switch) having a function equivalent to that of the foot switch 52.

In addition, the input device 50 can include, as the operating device, a known input device such as a keyboard, a mouse, a touch panel, a microphone, or a line-of-sight input device.

### Endoscopic Image Processing Apparatus

The endoscopic image processing apparatus 60 includes, as its hardware configuration, a processor, a main storage, an auxiliary storage, a communication unit, and the like. That is, the endoscopic image processing apparatus 60 has a so-called computer configuration as its hardware configuration. The processor is constituted by, for example, a CPU, a GPU (Graphics Processing Unit), an FPGA (Field Programmable Gate Array), a PLD (Programmable Logic Device), or the like. The processor of the endoscopic image processing apparatus 60 is an example of a processor in the endoscope system and the medical information processing apparatus according to the present invention. Note that the processor of the endoscopic image generation apparatus 40 and the processor of the endoscopic image processing apparatus 60 may share the functions of the processor in the endoscope system and the medical information processing apparatus according to the present invention. For example, an aspect can be adopted in which the endoscopic image generation apparatus 40 mainly includes a function of an “endoscope processor” that generates an endoscopic image, and the endoscopic image processing apparatus 60 mainly includes a function of a “CAD box (CAD: Computer Aided Diagnosis)” that performs image processing on the endoscopic image. However, the present invention may adopt an aspect different from such sharing of functions.

The main storage is constituted by, for example, a memory such as a RAM. The auxiliary storage is constituted by, for example, a non-transitory and tangible recording medium (memory) such as a flash memory, a ROM, or an EEPROM, and stores various programs (which may include the medical information processing program according to the present invention or a part thereof) to be executed by the processor, various kinds of data necessary for control, and the like. The communication unit is constituted by, for example, a communication interface that can be connected to a network. The endoscopic image processing apparatus 60 is communicably connected to the endoscope information management system 100 via the communication unit.

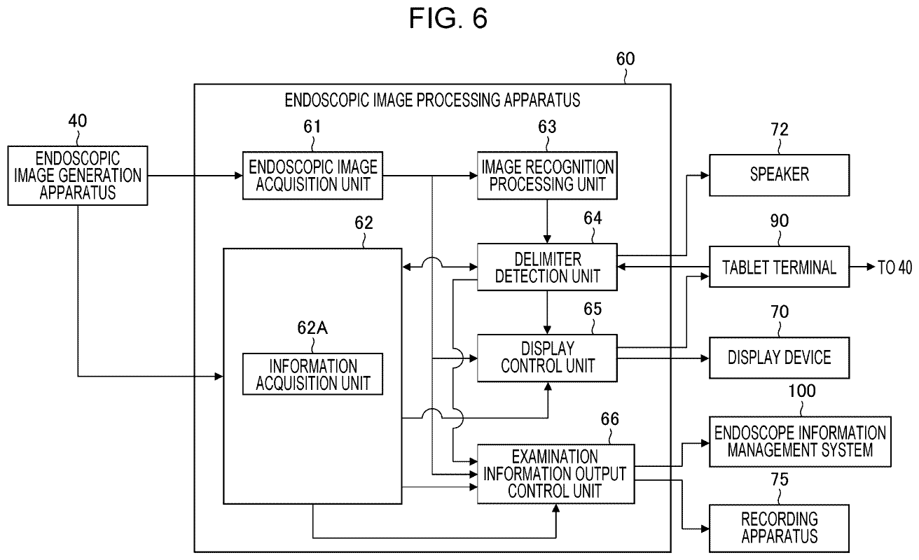

FIG. 6 is a block diagram illustrating main functions of the endoscopic image processing apparatus 60.

As illustrated in FIG. 6, the endoscopic image processing apparatus 60 mainly has functions of an endoscopic image acquisition unit 61, an input information acquisition unit 62, an image recognition processing unit 63, a delimiter detection unit 64, a display control unit 65, an examination information output control unit 66, and the like. These functions are implemented by the above-described processor executing a program (which may include the medical information processing program according to the present invention or a part thereof) stored in the auxiliary storage or the like.

### Endoscopic Image Acquisition Unit

The endoscopic image acquisition unit 61 acquires an endoscopic image from the endoscopic image generation apparatus 40. The image can be acquired in real time. That is, time-series medical images of a subject can be sequentially acquired (sequentially input) in real time.

### Input Information Acquisition Unit

The input information acquisition unit 62 (processor) acquires information that is input through the input device 50 and the endoscope 20. The input information acquisition unit 62 includes an information acquisition unit 62A that mainly acquires input information other than speech information.

Information that is input to the input information acquisition unit 62 through the input device 50 includes information (e.g., a result of speech recognition or a signal indicating a delimiter) that is input through the foot switch 52, a microphone 90A of the tablet terminal 90, a keyboard, a mouse, or the like that is not illustrated, or the like. In addition, the information that is input through the endoscope 20 includes information such as an instruction to start capturing an endoscopic image (moving image) and an instruction to capture a still image. As will be described later, in this embodiment, a user can input a signal indicating a delimiter for speech recognition, perform an operation of selecting a candidate for speech recognition, or the like through the microphone 90A or the foot switch 52. The input information acquisition unit 62 acquires operation information of the foot switch 52 through the endoscopic image generation apparatus 40.

### Image Recognition Processing Unit

The image recognition processing unit 63 (processor) performs image recognition on the endoscopic image acquired by the endoscopic image acquisition unit 61. The image recognition processing unit 63 can perform image recognition in real time (without a time delay from image acquisition to recognition).

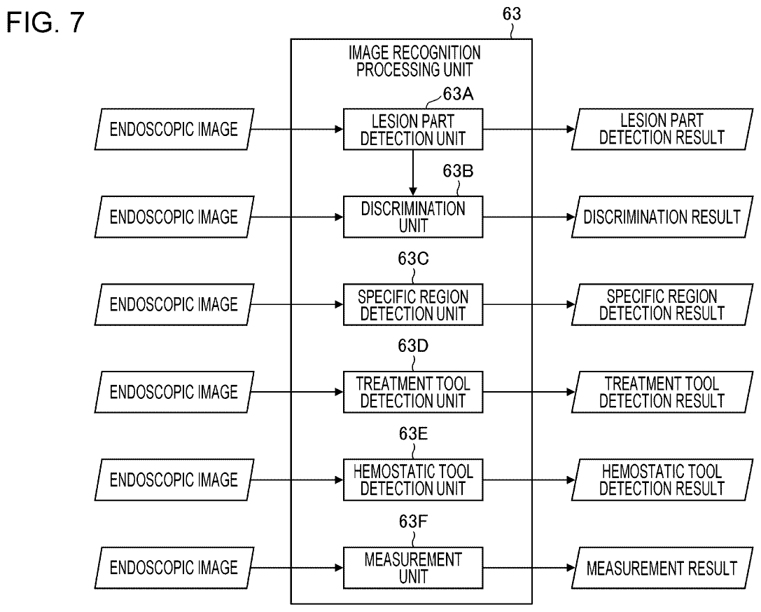

FIG. 7 is a block diagram illustrating main functions of the image recognition processing unit 63. As illustrated in FIG. 7, the image recognition processing unit 63 has functions of a lesion part detection unit 63A, a discrimination unit 63B, a specific region detection unit 63C, a treatment tool detection unit 63D, a hemostatic tool detection unit 63E, a measurement unit 63F, and the like. These units can be used for determining “whether a specific subject is included in the endoscopic image”. The “specific subject” is, for example, at least one of a lesion, a candidate lesion region, a landmark, or a post-treatment region, and may include a treatment tool or a hemostatic tool. In addition, the “specific subject” may be different depending on each unit of the image recognition processing unit 63 as described below.

The lesion part detection unit 63A detects a lesion part (lesion; example of “specific subject”) such as a polyp from the endoscopic image. The process for detecting a lesion part includes, in addition to a process for detecting a part that is definitely a lesion part, a process for detecting a part that may be a lesion part (e.g., benign tumor or dysplasia; candidate lesion region), a process for recognizing a region after treatment of a lesion (post-treatment region), a process for recognizing a part having a feature (e.g., redness) that may be directly or indirectly related to a lesion, and the like.

If the lesion part detection unit 63A determines that “a lesion part (specific subject) is included in the endoscopic image”, the discrimination unit 63B performs a discrimination process on the lesion part detected by the lesion part detection unit 63A (start of a discrimination mode). In this embodiment, the discrimination unit 63B performs the discrimination process of the lesion part such as a polyp detected by the lesion part detection unit 63A as neoplastic (NEOPLASTIC) or non-neoplastic (HYPERPLASTIC). Note that the discrimination unit 63B can be configured to start output of a discrimination result if a predetermined criterion is satisfied. As the “predetermined criterion”, for example, “a case where the reliability of the discrimination result (depending on conditions such as the exposure of the endoscopic image, the degree of focus, and blurring) or a statistical value thereof (e.g., a maximum or minimum within a predetermined period or an average) is greater than or equal to a threshold value” can be adopted, but another criterion may be used. A start of a discrimination mode and a start of output of a discrimination result can be used as a start delimiter (one of delimiters, other one of delimiters) when results of speech recognition are grouped.

The specific region detection unit 63C performs a process for detecting a specific region (landmark) in a luminal organ from the endoscopic image. For example, a process for detecting an ileocecal part of a large intestine, or the like is performed. The large intestine is an example of the luminal organ, and the ileocecal part is an example of the specific region. The specific region detection unit 63C may detect, for example, a hepatic flexure (right colon), a splenic flexure (left colon), a rectosigmoid, or the like. In addition, the specific region detection unit 63C may detect a plurality of specific regions.

The treatment tool detection unit 63D performs a process for detecting, from an endoscopic image, a treatment tool that appears in the image and determining the type of the treatment tool. The treatment tool detection unit 63D can be configured to detect a plurality of types of treatment tools such as biopsy forceps and a snare. Similarly, the hemostatic tool detection unit 63E performs a process for detecting a hemostatic tool such as a hemostatic clip and determining the type thereof. The treatment tool detection unit 63D and the hemostatic tool detection unit 63E may be configured by one image recognizer.

In a measurement mode, the measurement unit 63F measures a lesion, a candidate lesion region, a specific region, a post-treatment region, and the like (measures the shape, the dimension, and the like).

The units (e.g., the lesion part detection unit 63A, the discrimination unit 63B, the specific region detection unit 63C, the treatment tool detection unit 63D, the hemostatic tool detection unit 63E, and the measurement unit 63F) of the image recognition processing unit 63 can be configured by using an image recognizer (learned model) generated by machine learning. Specifically, the above-described units can be configured by an image recognizer (learned model) that performs learning by using a machine learning algorithm (or a derivative type thereof) such as a neural network (NN), a convolutional neural network (CNN), AdaBoost, or a Random Forest. In addition, as described above for the discrimination unit 63B, by setting a layer configuration of a network or the like as necessary, these units can output the reliability of the final output (e.g., the discrimination result and the type of the treatment tool) in combination. In addition, each of the above-described units may perform image recognition on all frames of the endoscopic image or may perform image recognition intermittently on some frames.

In the endoscope system 10, output of a recognition result of an endoscopic image from each of these units or output of a recognition result satisfying a predetermined criterion (e.g., a threshold value of reliability) may be a start delimiter or an end delimiter (trigger of speech input) of speech recognition, or a period during which such output is performed may be a period during which speech recognition is performed.

In addition, instead of configuring each unit constituting the image recognition processing unit 63 by an image recognizer (learned model), a configuration can also be adopted in which a feature quantity is calculated from an endoscopic image for some or all of the units, and detection or the like is performed by using the calculated feature quantity.

### Delimiter Detection Unit

The delimiter detection unit 64 (processor) detects a delimiter (an end delimiter when results of speech recognition are grouped; one of delimiters, other one of delimiters) with respect to the results of speech recognition. Specifically, the delimiter detection unit 64 can recognize, as the end delimiter, at least one of an end of detection of a specific subject in the endoscopic image (medical image), speech input of a first specific word/phrase to the microphone 90A (speech recognition device), continuation of a non-input state of speech input to the microphone 90A for a determined time or more, completion of speech input to all items to be subjected to speech recognition, completion of speech input to a specific item among the items to be subjected to speech recognition, acquisition of information indicating that an insertion length and/or an insertion shape of the endoscope has changed by a determined value or more, or a start or stop of an operation by a user of the endoscope system via an operating device (e.g., the foot switch 52 or an operating member provided in the operating unit 22). Details of speech recognition using these as delimiters will be described later.

Note that the delimiter detection unit 64 can determine, for example, at least one of a lesion, a candidate lesion region, a landmark, or a post-treatment region as the “specific subject”, but may also recognize a treatment tool or a hemostatic tool as the “specific subject”. In addition, the delimiter detection unit 64 can measure the insertion length and/or the insertion shape of the endoscope by using, for example, a colonoscope shape determination device connected to the endoscope system 10.

### Display Control Unit

The display control unit 65 (processor) controls display on the display device 70. Main display control performed by the display control unit 65 will be described below.

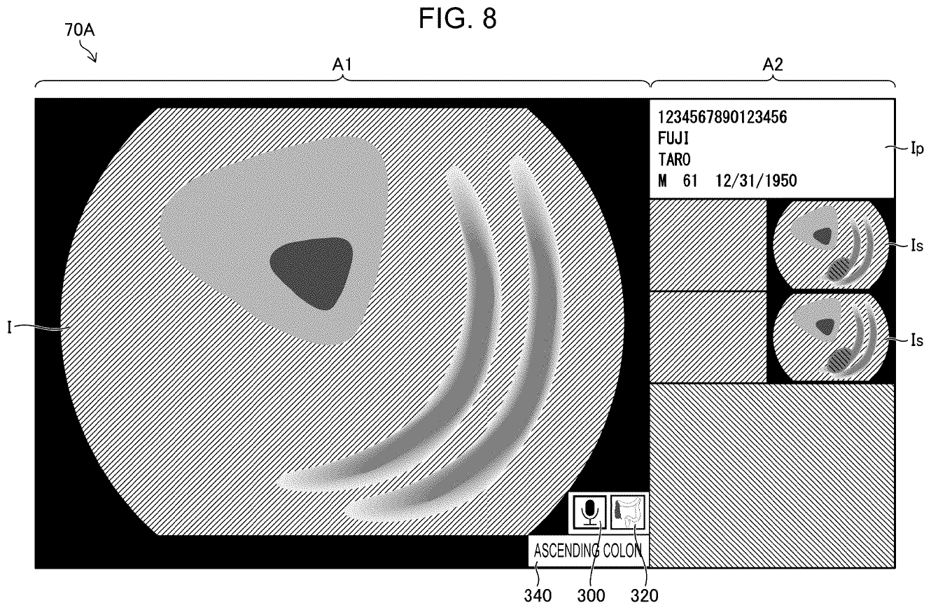

During an examination (during image capturing), the display control unit 65 causes the display device 70 to display the image (endoscopic image) captured by the endoscope 20 in real time (without a time delay). FIG. 8 is a diagram illustrating an example of a screen displayed during an examination. As illustrated in FIG. 8, an endoscopic image I (live view) is displayed in a main display region A1 set within a screen 70A. A sub-display region A2 is further set on the screen 70A, and various kinds of information on the examination are displayed therein. In the example illustrated in FIG. 8, an example in a case where information Ip on a patient and still images Is of the endoscopic image captured during the examination are displayed in the sub-display region A2 is illustrated. The still images Is are displayed, for example, in the order of being captured from the top to the bottom of the screen 70A. Note that if a specific subject such as a lesion is detected, the display control unit 65 may display the subject in an emphasized manner by using a bounding box or the like.

In addition, the display control unit 65 can display, on the screen 70A, an icon 300 indicating a state of speech recognition, an icon 320 indicating a site that is being imaged, and a display region 340 in which sites (e.g., ascending colon, transverse colon, and descending colon) that are imaging targets and results of speech recognition are displayed by characters in real time (without a time delay). In addition, if speech recognition is enabled, the display control unit 65 may display a message for encouraging speech input on the screen 70A.

The display control unit 65 can acquire and display information of sites by image recognition from the endoscopic image, input by a user via an operating device, an external device (e.g., an endoscope insertion shape observation device) connected to the endoscope system 10, or the like. Note that the display control unit 65 may cause a display 90E of the tablet terminal 90 or another display device to display various kinds of information.

### Examination Information Output Control Unit

The examination information output control unit 66 outputs examination information to the recording apparatus 75 and/or the endoscope information management system 100. In addition, the examination information output control unit 66 may output the examination information to a flash memory 90H or the database 210. The examination information may include, for example, an endoscopic image captured during an examination, a determination result of a specific subject, a result of speech recognition, information of a site, a treatment name, or a treatment tool, which is input during an examination, and the like. As will be described later, the examination information output control unit 66 can group and output these pieces of information. In addition, the examination information output control unit 66 can output the examination information, for example, for each lesion or each extracted test substance.

The examination information output control unit 66 can output, for example, an endoscopic image obtained by capturing an image of a lesion part or the like in association with a result of speech recognition or information of a site. In addition, if treatment is performed, the examination information output control unit 66 can also output information of the selected treatment name and information of a detected treatment tool in association with the endoscopic image, information of the site, the result of speech recognition, and the like. In addition, the examination information output control unit 66 can output an endoscopic image captured separately from the lesion part or the like to the recording apparatus 75 and/or the endoscope information management system 100 as appropriate. The examination information output control unit 66 may output the endoscopic image with information of the imaging date and time added thereto.

As will be described later, the examination information output control unit 66 can output the examination information by associating pieces of information with one another and grouping the pieces of information in accordance with delimiters of speech recognition.

### Recording Apparatus

The recording apparatus 75 (recording apparatus) includes various magneto-optical recording apparatuses and semiconductor memories, and a control device thereof, and can record an endoscopic image (moving image, still image), a result of image recognition, a result of speech recognition, examination information, report creation support information, and the like. These pieces of information may be recorded in a sub-storage of the endoscopic image generation apparatus 40 or the endoscopic image processing apparatus 60 or a recording apparatus included in the endoscope information management system 100, or may be recorded in a memory of the tablet terminal 90 or the database 210.

### Tablet Terminal

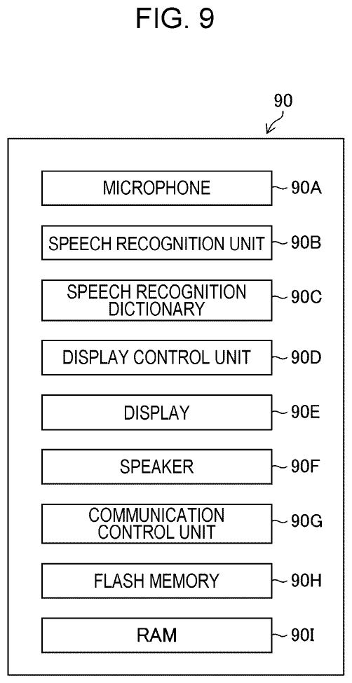

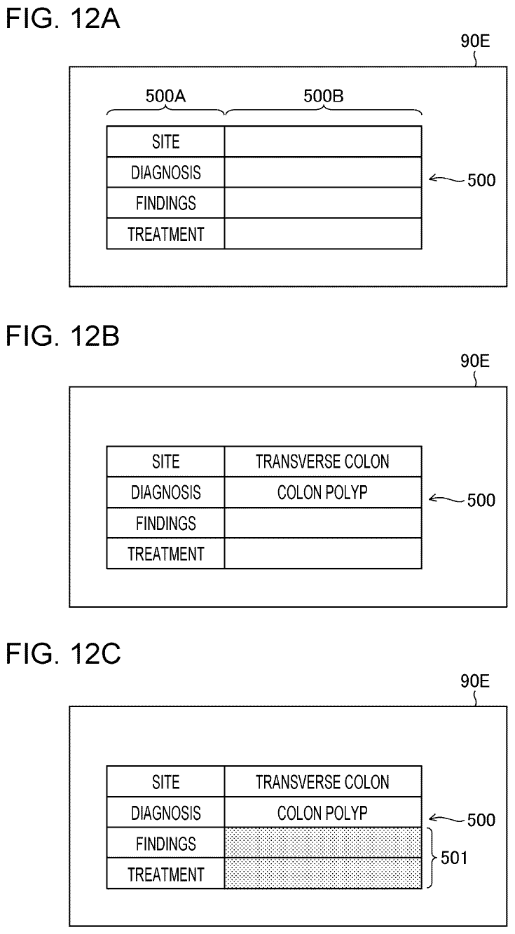

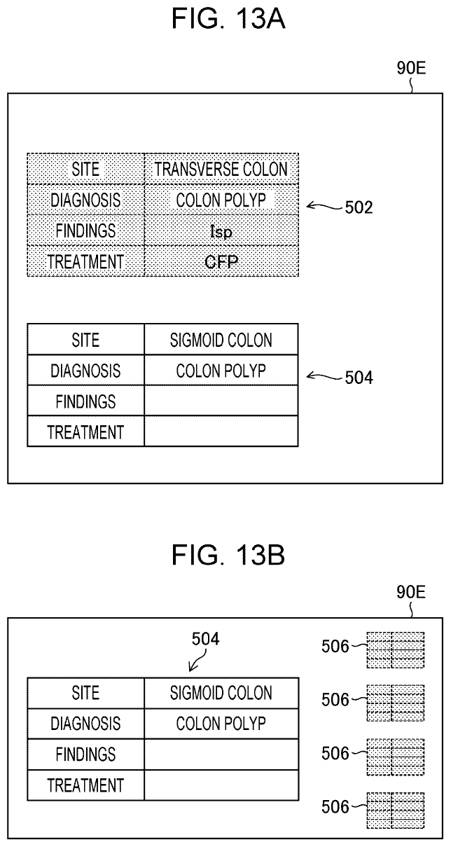

FIG. 9 is a diagram illustrating a configuration of the tablet terminal 90. As illustrated in FIG. 9, the tablet terminal 90 includes the microphone 90A (speech input device), a speech recognition unit 90B that recognizes speech that is input to the microphone 90A, and a speech recognition dictionary 90C used for speech recognition. The speech recognition dictionary 90C may include a plurality of dictionaries with different contents (e.g., dictionaries related to site information, findings information, treatment information, and hemostasis information). In addition, the tablet terminal 90 includes a display control unit 90D that controls display of a lesion information input box (item information and a result of speech recognition corresponding to the item information; see FIGS. 12A to 12C and FIGS. 13A and 13B) and the like, which will be described later, the display 90E (display device) on which the lesion information input box and the like are displayed, a speaker 90F (output device), and a communication control unit 90G, and can access the database 210 on the cloud 200 via the communication control unit 90G.

The speech recognition unit 90B performs speech recognition by referring to the speech recognition dictionary 90C. The speech recognition dictionary 90C may include a plurality of dictionaries having different features (e.g., target sites), and the image recognition processing unit 63 may recognize an imaged site of an endoscopic image, and the speech recognition unit 90B may select an appropriate speech recognition dictionary based on the recognition result.

Note that although a case where the tablet terminal 90 includes the microphone 90A and the speaker 90F is described in FIG. 9, in addition to or instead of these devices, an external microphone and/or speaker or a headset (speech input device, output device) including a microphone and a speaker may be used.

In addition, the tablet terminal 90 functions as an interface for speech recognition. For example, speech recognition customization settings for each of users can be stored in the flash memory 90H or the like and displayed on the display 90E in accordance with a user operation, usage guidance can be displayed on the display 90E, or an operation history of an application (program) for the tablet terminal 90 can be collected and displayed. In addition, the tablet terminal 90 can acquire or update an application or data by connecting to the Internet or a cloud via the communication control unit 90G. The speech recognition unit 90B may learn speech recognition in accordance with a feature of a user's utterance.

The functions of the tablet terminal 90 described above can be implemented by using a processor such as a CPU. At the time of processing by the processor, a program (the medical information processing program according to the present invention or a part thereof (mainly, a part related to speech recognition)) stored in the flash memory 90H (an example of a non-transitory and tangible recording medium) is referred to, and a RAM 90I is used as a temporary storage area or a work area.

In the endoscope system 10 according to the first embodiment, instead of or in addition to the tablet terminal 90, a device such as a desktop or notebook computer or a smartphone may be used.

### Sharing of Functions in Endoscope System

Note that “how the functions implemented in the endoscope system 10 are shared by the endoscopic image generation apparatus 40, the endoscopic image processing apparatus 60, and the tablet terminal 90” is not limited to the above-described example. For example, what is described above as a function of the endoscopic image generation apparatus 40 or the endoscopic image processing apparatus 60 may be executed by the tablet terminal 90, and, conversely, what is described above as a function of the tablet terminal 90 may be executed by the endoscopic image generation apparatus 40 or the endoscopic image processing apparatus 60. In addition, as in a second embodiment described later, all the functions may be executed by the endoscopic image generation apparatus 40 and the endoscopic image processing apparatus 60 without providing the tablet terminal 90.

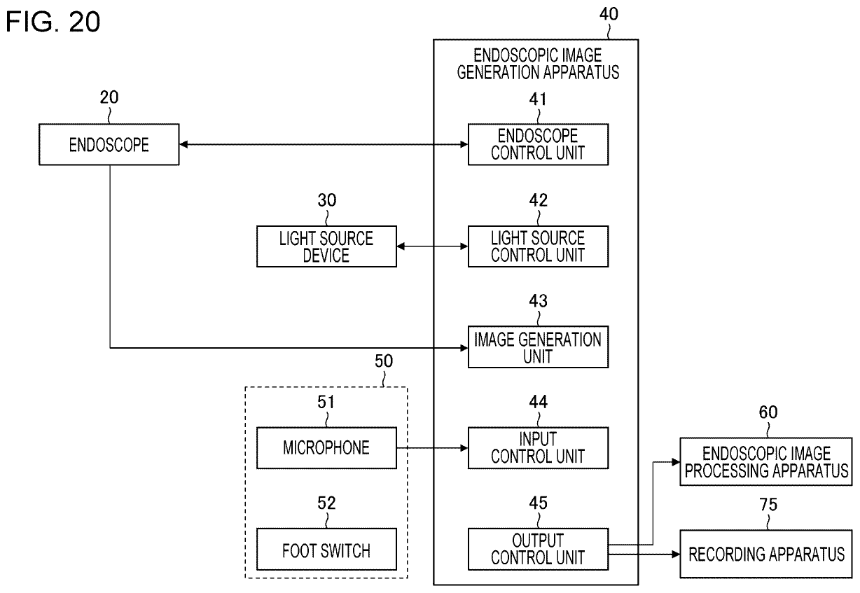

Note that although a case where speech input is performed using the microphone 90A is described in the first embodiment, the input device 50 may include a microphone instead of the microphone 90A or in addition to the microphone 90A (see the second embodiment described later and FIG. 20).

### Speech Recognition in Endoscope System

Speech recognition and recording of a result thereof in the endoscope system 10 having the above-described configuration will be described below.

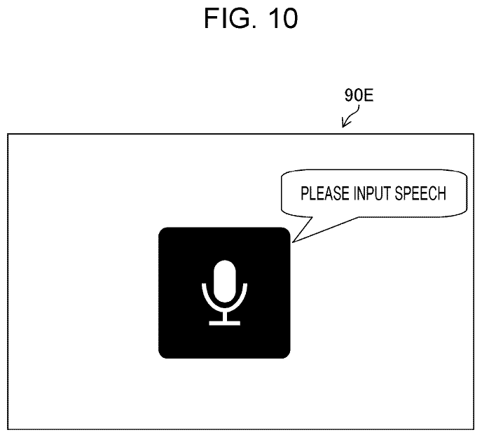

When speech input and speech recognition are enabled, or when capturing of time-series endoscopic images starts, the delimiter detection unit 64 (processor) can detect these as a start delimiter (one of delimiters) of grouping. In response to such detection, the display control unit 90D (processor) can cause an output device to output a message for encouraging speech input for the endoscopic image. Specifically, the display control unit 90D may display a message as illustrated in FIG. 10 on the display 90E (output device) of the tablet terminal 90, or may output a voice message from a speaker 72 (output device) or the speaker 90F (output device). By such a message being output, a user can easily grasp that speech recognition is enabled.

Note that the speech recognition unit 90B may start speech recognition and grouping results thereof after output of a message, or may automatically start speech recognition and grouping results thereof when capturing of an endoscopic image (time-series medical images) starts (in this case, the delimiter detection unit 64 can detect a start of image capturing as a “start delimiter of grouping”).

### Grouping of Results of Speech Recognition

The delimiter detection unit 64 (processor) detects a delimiter (an end delimiter of speech recognition; delimiter) of results of speech recognition. If the delimiter detection unit 64 detects a start delimiter (one of delimiters) and then re-detects an end delimiter (other one of delimiters) corresponding to the start delimiter at a time later than the time at which the start delimiter is detected, the examination information output control unit 66 (processor) groups and records results of speech recognition during a period from the start delimiter to the end delimiter in the recording apparatus 75 and/or the flash memory 90H (recording apparatus).

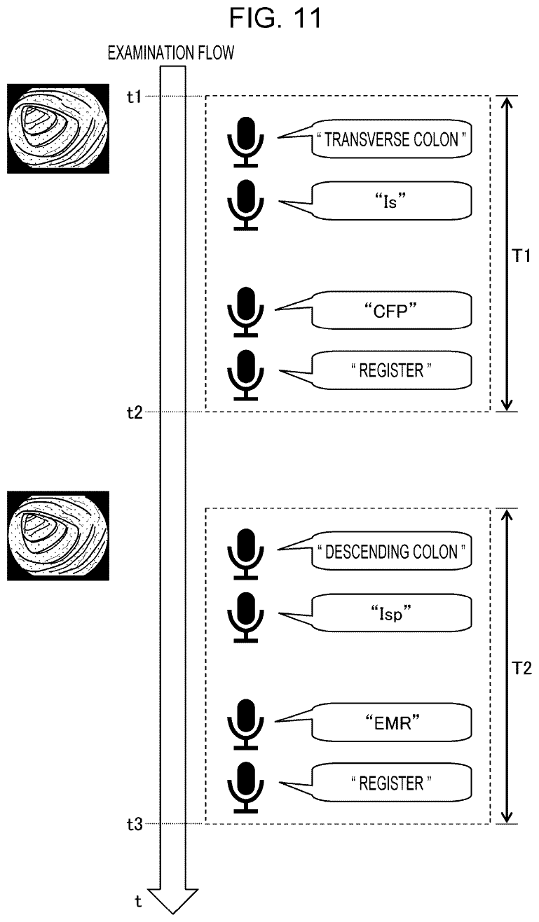

FIG. 11 is a diagram illustrating a state in which results of speech recognition are grouped and recorded. FIG. 11 illustrates an example of grouping speech input and speech recognition of the word “register” (first specific word/phrase) as an end delimiter (delimiter), and the examination information output control unit 66 records each of results of speech recognition during a period T1 and results of speech recognition during a period T2 as one group. The word “register” is an example of the first specific word/phrase, and another word/phrase such as “confirm” may be used. The word “register” itself does not have to be grouped.

Note that specific aspects of the “grouping” include recording a plurality of results of speech recognition in one file or folder (which may be recorded in units of lesion information input boxes described later), adding a link of another result of speech recognition to a result of speech recognition, and the like.

In the example in FIG. 11, the period T1 and the period T2 are speech recognition periods for different lesions. In addition, in the following drawings, figures of microphones indicate timings of speech input and speech recognition, and speech recognition is also performed in accordance with speech input.

For the period T1, it is assumed that a start delimiter (one of delimiters) is detected at time T1 in response to image capturing being started, speech input being enabled, or the like. In addition, at time t2 later than time t1 at which the start delimiter is detected, an end delimiter corresponding to the start delimiter (speech input of the word “register”; other one of delimiters corresponding to the one of delimiters) is detected. In addition, for the period T2, after the end delimiter is detected at time t2 in the period T1, the end delimiter (the word “register”) is re-detected at time t3 later than time t2, and the results of speech recognition during the period from time t2 to time t3 are grouped. That is, in the example in FIG. 11, the speech input of the word “register” at time t2 is an end delimiter of the period T1 and is also a start delimiter of the period T2.

According to the first embodiment, by grouping the results of speech recognition, it is possible to easily grasp related results of speech recognition. The results of speech recognition grouped in this manner can be utilized for report creation or the like.

### Display of Lesion Information Input Box

Upon a start of speech recognition, as illustrated in FIGS. 12A to 12C, the display control unit 90D (processor) causes the display 90E (display device) to display a lesion information input box (item information indicating items to be subjected to speech recognition) and results of speech recognition corresponding to the item information. FIG. 12A is an example (non-input state) of displaying a lesion information input box 500, and the lesion information input box 500 is constituted by a region 500A indicating item information and a region 500B indicating results of speech recognition corresponding to the item information. In the examples in FIGS. 12A to 12C, the item information includes site, diagnosis, findings, and treatment (one set of pieces of item information). In this manner, the item information preferably includes at least one of site, diagnosis, findings, or treatment. In addition, FIG. 12B illustrates a state in which speech input and speech recognition are performed for a site and diagnosis among pieces of the item information. With such a lesion information input box, a user can easily grasp an item that is a target of speech recognition and an input state thereof.

FIG. 12C illustrates an example in which a region 501 for displaying items that have not been input is grayed out (an aspect of identification display). By the identification display being performed in this manner, a user can easily grasp the items that have not been input. Note that the display control unit 90D can display the lesion information input box 500 (item information) and the results of speech recognition in real time (without a time delay).

Note that the display control unit 90D can cause a display device, which is different from the display device that displays the time-series endoscopic images, to display the results of speech recognition.

### Change of Display Manner of Lesion Information Input Box

The above-described lesion information input box is displayed, and information is input thereto for each lesion (an example of a region of interest), and, if a plurality of lesions are found in an examination, a plurality of lesion information input boxes are displayed, and information is input thereto in correspondence with the lesions. In such a case, upon detection of an end delimiter (delimiter) of the grouping, the display control unit 90D can change a display manner of the item information and the results of speech recognition on the display 90E (display device) (e.g., to be less identifiable). FIGS. 13A and 13B are diagrams illustrating examples of such a change of the display manner. In the example illustrated in FIG. 13A, the display control unit 90D grays out a lesion information input box 502 in which grouping is determined while changing the frame to dotted lines, and in the example illustrated in FIG. 13B, the display control unit 90D further displays lesion information input boxes 506 in which grouping is determined as thumbnail images. In addition to these manners, the display control unit 90D may display a lesion information input box in which grouping is determined as an icon, or may delete the lesion information input box. By the display manner being changed in this manner, a user can easily grasp the lesion information input box that is currently an input target.

### Grouping of Results of Speech Recognition and Image

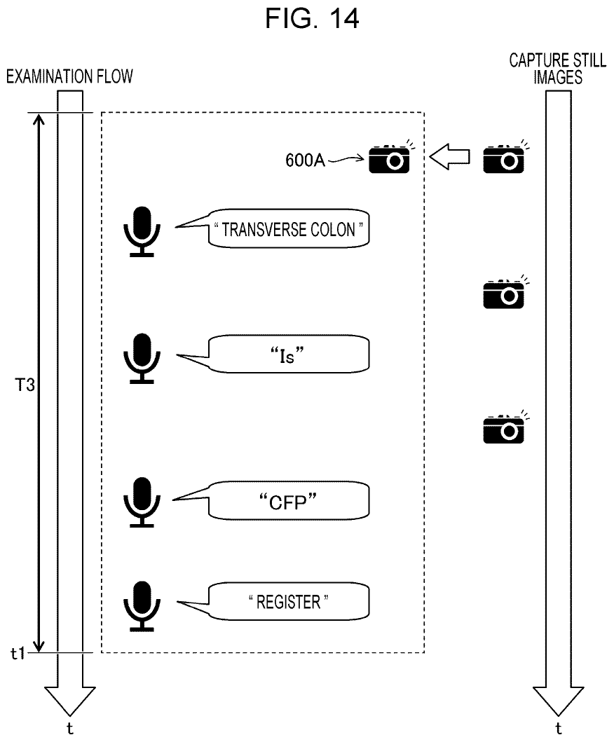

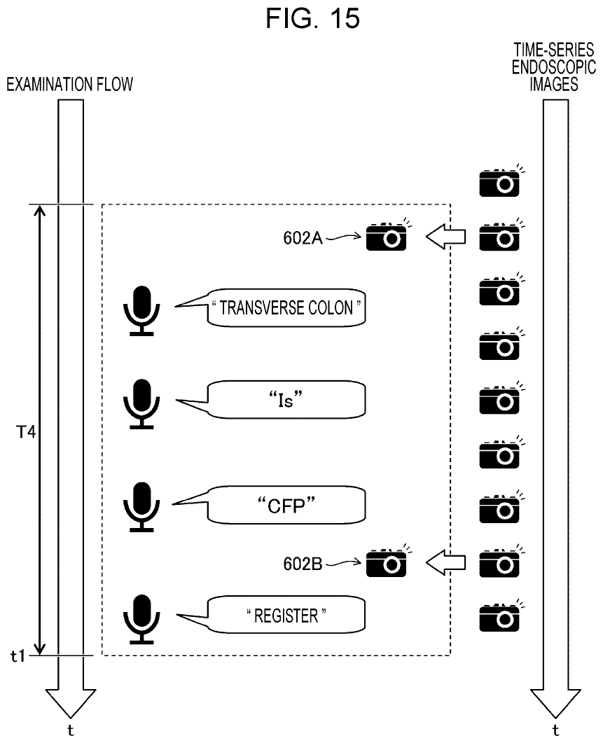

In the present invention, it is possible to group an image selected from medical images captured by an endoscope during a period until an end delimiter (delimiter) of grouping is detected, together with results of speech recognition and record the image in a recording apparatus (e.g., the recording apparatus 75 or the flash memory 90H). FIG. 14 is a diagram illustrating a state in which an image is also grouped. In the example illustrated in FIG. 14, the examination information output control unit 66 selects a still image 600A from among three still images (captured images captured separately from the time-series medical images; indicated by symbols of cameras in FIGS. 14 and 15) captured during a period T3, and groups the still image 600A together with the results of speech recognition. Note that the speech input of the word “register” at time t1 is an end delimiter of the period T3, and an end delimiter in a previous period or the like can be a start delimiter of the period T3 (the same applies to FIGS. 15, 16, and 17 described later).

FIG. 15 is another diagram illustrating a state in which images are also grouped. In the example illustrated in FIG. 15, the examination information output control unit 66 selects images 602A and 602B from frame images constituting time-series medical images captured during a period T4, and groups the images 602A and 602B together with results of speech recognition.

The examination information output control unit 66 can automatically (without depending on a user operation) select an image to be grouped together with the results of speech recognition based on a determined condition. For example, the examination information output control unit 66 can select a still image captured at a determined timing. The “still image captured at a determined timing” is a still image initially captured during the period T3 in the example in FIG. 14, but may be a still image captured at another timing, such as a timing before and/or after treatment. In addition, the examination information output control unit 66 may select an image based on image quality. For example, the examination information output control unit 66 can select an image with little bokeh or blur or an image whose brightness falls within a determined range. In addition, the examination information output control unit 66 may select an image based on a user operation. The examination information output control unit 66 may select an image concurrently with speech recognition, or may select an image after grouping of the results of speech recognition is completed.

### Variation of End Delimiter of Grouping

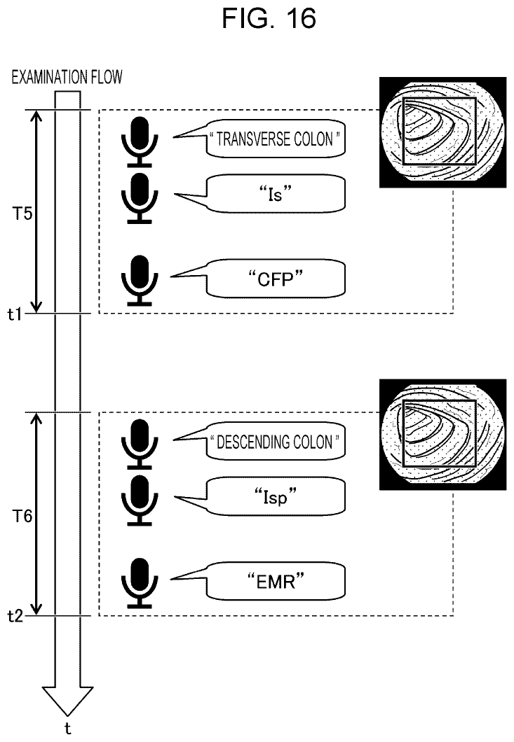

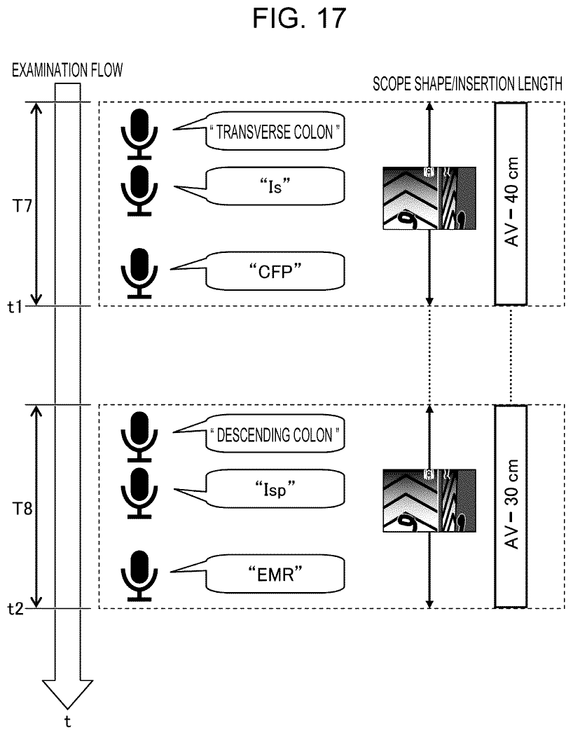

In the above example, a case has been described in which speech recognition of a specific word/phrase (first specific word/phrase) of grouping is set as an end delimiter (delimiter) of grouping, but the delimiter detection unit 64 (processor) can also detect other information as the end delimiter. FIG. 16 is an example in which the end delimiter is that the image recognition processing unit 63 ends detection of a specific subject (here, a lesion) (different lesions are detected during a period T5 and a period T6), and FIG. 17 is an example of using an end delimiter based on the insertion length and/or the insertion shape of the endoscope 20 (endoscope). In the example in FIG. 17, for example, during a period T7 and a period T8 in which the insertion shapes of the scope are similar and the change in the insertion lengths stagnates, the delimiter detection unit 64 can determine that “observation or treatment for a specific lesion is being performed” (an end delimiter is not detected) and, if the insertion length and/or the insertion shape change by an amount greater than or equal to a predetermined criterion (at an end of the period T7), can determine that “observation or treatment for a specific lesion has ended” (an “end delimiter is detected”). Note that the insertion length and/or the insertion shape of the endoscope 20 can be measured by, for example, connecting a colonoscope shape determination device to the endoscope system 10.

In the examples described above with reference to FIGS. 16 and 17, a user does not need to perform the “speech input of a specific word/phrase” described above with reference to FIG. 11.

Including the examples described above with reference to FIGS. 11, 16, and 17, the delimiter detection unit 64 can detect, as the end delimiter, at least one of an end of detection of a specific subject (e.g., at least one of a lesion, a candidate lesion region, a landmark, or a post-treatment region) in the time-series endoscopic images (medical images), speech input of the first specific word/phrase to the microphone 90A (speech recognition device), continuation of a non-input state of speech input to the microphone 90A for a determined time or more, completion of speech input to all items (site, diagnosis, findings, and treatment in the above example) to be subjected to speech recognition, completion of speech input to a specific item (e.g., treatment) among the items to be subjected to speech recognition, acquisition of information indicating that the insertion length and/or the insertion shape of the endoscope has changed by a determined value or more, or a start or stop of an operation by a user of the endoscope system via an operating device (e.g., the foot switch 52). If the detection accuracy of the end delimiter is low with only one piece of information, the delimiter detection unit 64 may combine a plurality of pieces of information among these pieces of information to increase the detection accuracy as the end delimiter.

### Start Delimiter of Grouping

Although the above aspect mainly defines the end delimiter of grouping and describes a case where an end delimiter of a previous period is a start delimiter of a subsequent period, an explicit start delimiter of grouping may be used as in the case of the end delimiter. In this case, the delimiter detection unit 64 detects a start delimiter (delimiter) of speech recognition during capturing (inputting) of an endoscopic image. If a start delimiter (delimiter) is detected, the speech recognition unit 90B may output a message for encouraging speech input, as in the example in FIG. 10.

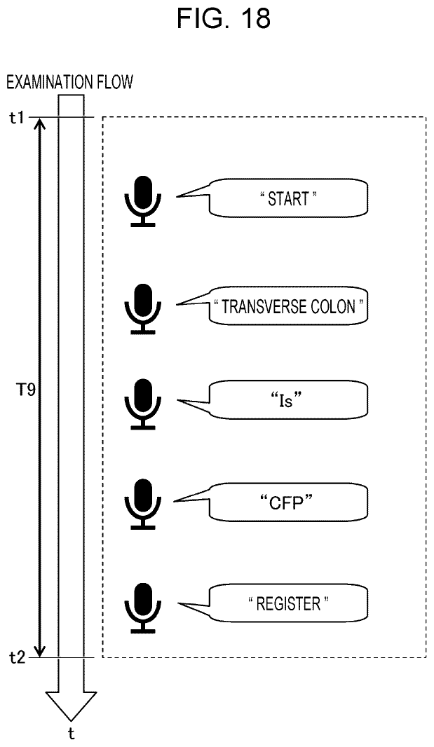

FIG. 18 is a diagram illustrating a state in which results of speech recognition during a period from a start delimiter to an end delimiter are grouped. In this example, the speech recognition unit 90B groups and records, in a recording apparatus (the recording apparatus 75 and/or the flash memory 90H), results of speech recognition (“transverse colon”, “Is”, and “CFP”) during a period T9 from when the delimiter detection unit 64 detects the word “start” (an example of a second specific word/phrase; start delimiter) at time t1 until when the delimiter detection unit 64 detects the word “register” (an example of the first specific word/phrase; end delimiter) at time t2 later than time t1. Note that the word “start” is an example of a second specific word/phrase, and another word may be used.

In addition to speech input of a specific word/phrase, for example, the delimiter detection unit 64 can detect a determination result (detection result) indicating a start of detection of a specific subject in an endoscopic image as a start delimiter of grouping, and, in this case, can use the output of the lesion part detection unit 63A as the determination result. In addition, the delimiter detection unit 64 may detect, as the start delimiter, a start of a discrimination mode for the specific subject, a start of output of a discrimination result for the specific subject, a start of a measurement mode for the specific subject, or the like (in this case, output of the discrimination unit 63B can be used as the discrimination result), or may detect, as the start delimiter, an instruction to start capturing time-series medical images, input of a wake word (an example of the second specific word/phrase) to the microphone 90A (speech input device), an operation of the foot switch 52, a user operation on another operating device (e.g., a colonoscope shape determination device) connected to the endoscope system, or the like. The speech recognition unit 90B may set the speech recognition dictionary 90C in accordance with the start delimiter.

### Second Embodiment

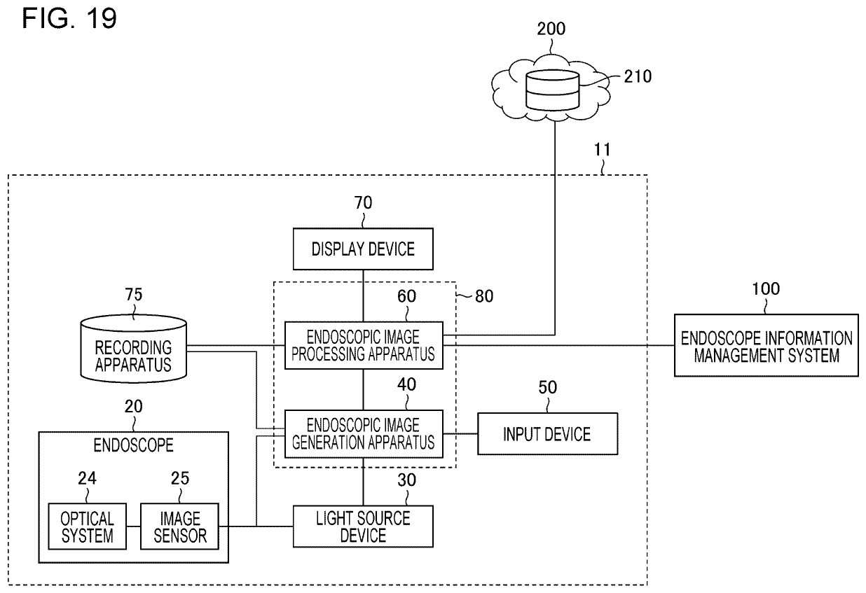

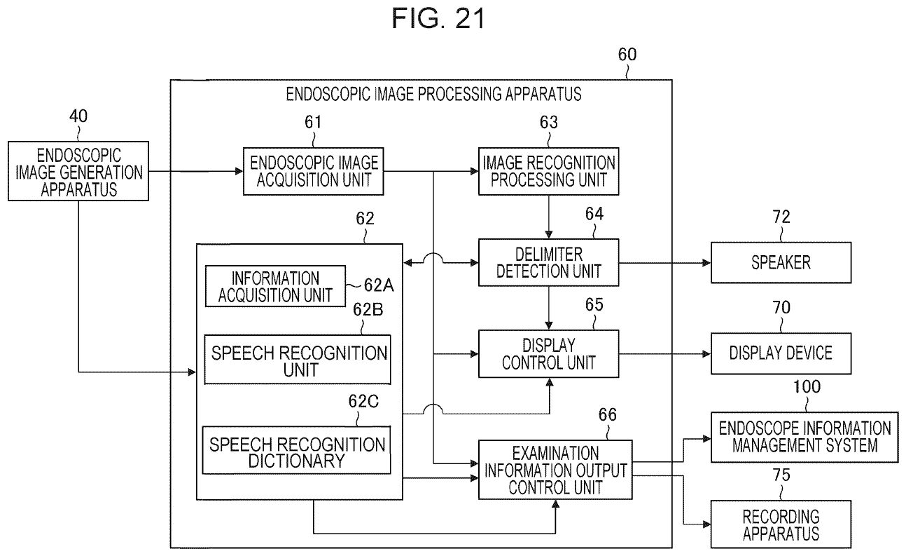

FIG. 19 is a diagram illustrating a configuration of an endoscope system 11 according to a second embodiment. In addition, FIG. 20 is a diagram illustrating a configuration of the endoscopic image generation apparatus 40 in the second embodiment, and FIG. 21 is a diagram illustrating a configuration of the endoscopic image processing apparatus 60 in the second embodiment. As illustrated in these drawings, in the second embodiment, the functions of the tablet terminal 90 in the first embodiment are implemented by the endoscopic image generation apparatus 40 and the endoscopic image processing apparatus 60. A user performs speech input via a microphone 51 of the input device 50, and a speech recognition unit 62B of the input information acquisition unit 62 performs speech recognition by using a speech recognition dictionary 62C.

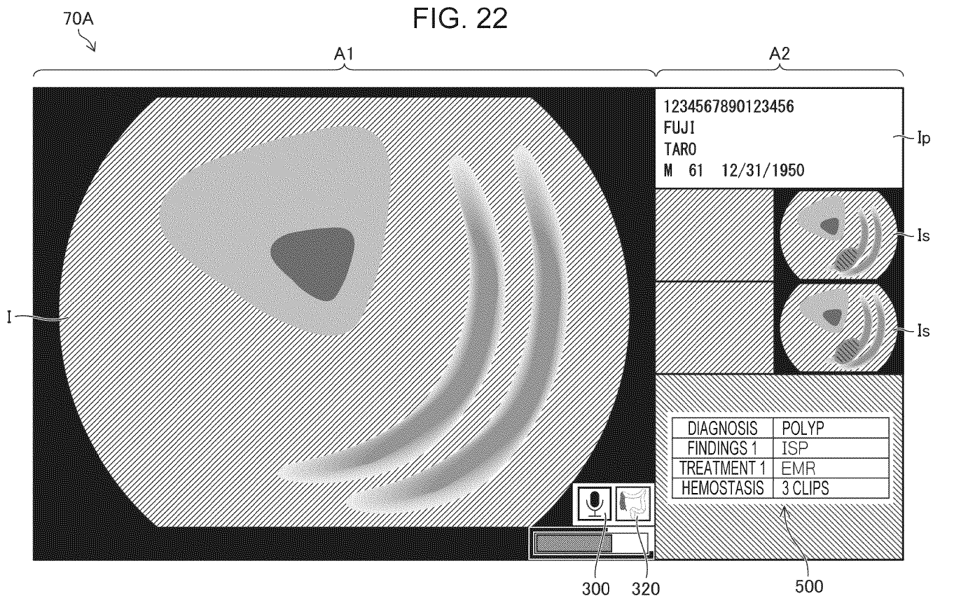

In the second embodiment, grouping of results of speech recognition or grouping of results of speech recognition and images can be performed in the same manner as in the first embodiment, and thus, a user can easily grasp related results of speech recognition. FIG. 22 is a diagram illustrating an example of results of speech recognition in the second embodiment, and illustrates a state in which the lesion information input box 500 (item information and results of speech recognition) is displayed on the screen 70A of the display device 70.

### Application to Endoscope for Upper Digestive Tract

Although the above embodiments have described a case where the present invention is applied to an endoscope system for the lower digestive tract, the present invention is also applicable to an endoscope for an upper digestive tract.

Although the embodiments of the present invention have been described above, the present invention is not limited to the above-described aspects, and various modifications can be made without departing from the spirit of the present invention.

### REFERENCE SIGNS LIST

1 endoscopic image diagnosis support system
10 endoscope system
11 endoscope system
20 endoscope
21 insertion part
21A tip part
21B bending part
21C soft part
21a observation window
21b illumination window
21c air/water supply nozzle
21d forceps outlet
22 operating unit
22A angle knob
22B air/water supply button
22C suction button
22D forceps insertion port
23 connection part
23A cord
23B light guide connector
23C video connector
24 optical system
25 image sensor
30 light source device
40 endoscopic image generation apparatus
41 endoscope control unit
42 light source control unit
43 image generation unit
44 input control unit
45 output control unit
50 input device
51 microphone
52 foot switch
60 endoscopic image processing apparatus
61 endoscopic image acquisition unit
62 input information acquisition unit
62A information acquisition unit
62B speech recognition unit
62C speech recognition dictionary
63 image recognition processing unit
63A lesion part detection unit
63B discrimination unit
63C specific region detection unit
63D treatment tool detection unit
63E hemostatic tool detection unit
63F measurement unit
64 delimiter detection unit
65 display control unit
66 examination information output control unit
70 display device
70A screen
72 speaker
75 recording apparatus
80 medical information processing apparatus
90 tablet terminal
90A microphone
90B speech recognition unit
90C speech recognition dictionary
90D display control unit
90E display
90F speaker
90G communication control unit
90H flash memory
90I RAM
100 endoscope information management system
200 cloud
210 database
300 icon
320 icon
340 display region
500 lesion information input box
500A region
500B region
501 region
502 lesion information input box
506 lesion information input box
600A still image
602A image
602B image
A1 main display region
A2 sub-display region
I endoscopic image
Ip information
Is still image
t1 time
12 time
t3 time
T1 period
T2 period
T3 period
T4 period
T5 period
T6 period
T7 period
T8 period
T9 period

What is claimed is:

1. An endoscope system comprising:
a speech recognition device configured to receive input of speech and perform speech recognition;
an endoscope configured to acquire a medical image of a subject; and
a processor, wherein
the processor is configured to:
cause the endoscope to capture time-series medical images of the subject;
detect delimiters of results of the speech recognition during capturing of the time-series medical images; and
group and record, in a recording apparatus, the results of the speech recognition, the results being obtained during a period from detection of one of the delimiters until detection of an other one of the delimiters corresponding to the one of the delimiters at a time later than a time at which the one of the delimiters is detected.

2. The endoscope system according to claim 1, wherein
the processor is configured to cause a display device to display item information indicating an item to be subjected to speech recognition and a result of the speech recognition corresponding to the item information if the speech recognition is started.

3. The endoscope system according to claim 2, wherein the processor is configured to record, in the recording apparatus, the results of the speech recognition corresponding to one set of pieces of the item information as one group.

4. The endoscope system according to claim 2, wherein the processor is configured to:
continue to display the item information and the result of the speech recognition from detection of the one of the delimiters until detection of the other one of the delimiters; and
change a display manner of the item information and the result of the speech recognition on the display device if the other one of the delimiters is detected.

5. The endoscope system according to claim 2, wherein
the processor is configured to cause the display device to display the item information and the result of the speech recognition in real time.

6. The endoscope system according to claim 2, wherein the item information includes at least one of diagnosis, findings, treatment, or hemostasis.

7. The endoscope system according to claim 1, wherein the processor is configured to detect the one of the delimiters as a start delimiter of grouping and detect the other one of the delimiters as an end delimiter of the grouping.

8. The endoscope system according to claim 7, wherein the processor is configured to group the results of the speech recognition during a period from detection of the end delimiter until re-detection of the end delimiter at a time later than a time at which the end delimiter is detected.

9. The endoscope system according to claim 7, wherein
the processor is configured to detect, as the end delimiter, at least one of an end of detection of a specific subject in the medical image, speech input of a first specific word/phrase to the speech recognition device, continuation of a non-input state of speech input to the speech recognition device for a determined time or more, completion of speech input to all items to be subjected to speech recognition, completion of speech input to a specific item among the items to be subjected to speech recognition, acquisition of information indicating that an insertion length and/or an insertion shape of the endoscope has changed by a determined value or more, or a start or stop of an operation by a user of the endoscope system via an operating device.

10. The endoscope system according to claim 7, wherein
the processor is configured to detect, as the start delimiter, at least one of a start of detection of a specific subject in the medical image, speech input of a second specific word/phrase to the speech recognition device, input by a user of the endoscope system via an operating device, a start of a discrimination mode for the specific subject, a start of output of a discrimination result for the specific subject, or a start of a measurement mode for the specific subject.

11. The endoscope system according to claim 9, wherein the processor is configured to determine at least one of a lesion, a candidate lesion region, a landmark, or a post-treatment region as the specific subject.

12. The endoscope system according to claim 9, wherein the processor is configured to recognize the specific subject by using an image recognizer generated by machine learning.

13. The endoscope system according to claim 8, wherein the processor is configured to cause an output device to output a message for encouraging speech input for the medical image if the start delimiter is detected.

14. The endoscope system according to claim 1, wherein
the processor is configured to cause an image selected from the medical images captured by the endoscope during a period from detection of the one of the delimiters until detection of the other one of the delimiters to be grouped and recorded together with the results of the speech recognition.

15. The endoscope system according to claim 1, wherein the processor is configured to cause an image selected from frame images constituting the time-series medical images and/or an image selected from captured images captured separately from the time-series medical images to be grouped and recorded together with the results of the speech recognition.

16. The endoscope system according to claim 1, wherein the processor is configured to cause a display device to display the time-series medical images and a different display device to display the results of the speech recognition.

17. A medical information processing method to be executed by an endoscope system comprising: a speech recognition device configured to receive input of speech and perform speech recognition; an endoscope configured to acquire a medical image of a subject; and a processor, the medical information processing method, comprising:
causing, by the processor, the endoscope to capture time-series medical images of the subject;
detecting, by the processor, delimiters of results of the speech recognition during capturing of the time-series medical images; and
grouping and recording in a recording apparatus, by the processor, the results of the speech recognition, the results being obtained during a period from detection of one of the delimiters until detection of an other one of the delimiters corresponding to the one of the delimiters at a time later than a time at which the one of the delimiters is detected.

18. A non-transitory, computer-readable tangible recording medium which records thereon a medical information processing program causing an endoscope system to execute a medical information processing method, the endoscope system comprising: a speech recognition device configured to receive input of speech and perform speech recognition; an endoscope configured to acquire a medical image of a subject; and a processor, wherein
the medical information processing method comprises:
causing, by the processor, the endoscope to capture time-series medical images of the subject;
detecting, by the processor, delimiters of results of the speech recognition during capturing of the time-series medical images; and
grouping and recording in a recording apparatus, by the processor, the results of the speech recognition, the results being obtained during a period from detection of one of the delimiters until detection of an other one of the delimiters corresponding to the one of the delimiters at a time later than a time at which the one of the delimiters is detected.
